(19) United States
(12) Patent Application Publication
(10) Pub. No.: US 2015/0034074 A1
(43) Pub. Date: Feb. 5, 2015

(54) Vulvar Varicosity Supporter

(71) Applicant: Lev Tripolsky, Redwood City, CA (US)

(72) Inventor: Lev Tripolsky, Redwood City, CA (US)

(21) Appl. No.: 14/449,982

(22) Filed: Aug. 1, 2014

Related U.S. Application Data
(60) Provisional application No. 61/861,859, filed on Aug. 2, 2013.

(51) Int. Cl. A61F 5/28; A41C 1/10

(52) U.S. Cl. CPC A61F 5/28; A41C 1/10
USPC 128/98.1

(57) ABSTRACT

A vulvar varicosity supporter that firmly attaches to an existing maternity support belt or back support belt in such a way that it can provide significant force on the vulva, and, in an embodiment, comprises a pocket for an insertable pad that can increase the pressure on the vulva.

### CROSS-REFERENCE TO RELATED APPLICATIONS

The present application takes priority from Provisional App. No. 61/861,859, filed Aug. 2, 2013, which is incorporated herein by reference.

### BACKGROUND

Vulvar varicose veins are a painful problem afflicting pregnant women in the later stages of pregnancy. While they are typically self-limiting and resolve after the pregnancy is over, they are still a significant source of discomfort during pregnancy. There is a need for devices and methods to relieve this discomfort.

Various types of braces exist to provide upward pressure on the vulva to relieve the discomfort caused by varicose veins. Typically, the designs are insufficiently sturdy, so that they do not provide enough pressure, or involve too many complicated straps and attachments, so that they are difficult to put on or take off. Since many pregnant women afflicted by vulvar varicose veins also suffer from back pain or other problems associated with the weight and size of the enlarged abdomen, they often wear a maternity support belt that supports the abdomen and back. A woman who wears both a maternity support belt and a vulvar brace will therefore have to struggle with two complicated support garments. Also, a maternity support belt is stronger and sturdier than a dedicated belt for a vulvar varicosity supporter, and thus can allow it to provide greater pressure on the vulva. A need therefore exists for a vulvar brace that easily attaches to a maternity support belt and provides sufficient and comfortable support to vulvar varicosities.

### SUMMARY OF THE INVENTION

An object of the present invention is to provide a support brace for vulvar varicosities that attaches to an existing maternity support belt or back support belt.

Another object of the present invention is to provide a support brace for vulvar varicosities that provides a firmer support than existing support braces.

Another object of the present invention is to provide a support brace for vulvar varicosities that provides an adjustable level of support.

The present invention is a support brace for vulvar varicosities, comprising a vulvar support which comfortably fits over the patient's vulvar area and straps attached to the vulvar support both at the front and at the back. The straps comprise fasteners that attach to a maternity support belt. The fasteners can be Velcro fasteners, snaps, buttons, hooks, zippers, or any other fasteners that can firmly attach a strap to a belt.

The straps can be adjustable in length and can be tightened while the support brace is worn. This enables the patient to adjust the pressure of the support brace while it is in use. In an embodiment, there are two straps attached to the front of the vulvar support and two straps attached to the back. In one embodiment, there is a connecting strap attached to each of the back straps to connect them and to prevent them from moving too far apart while the patient is sitting or walking or bending over.

The vulvar support is preferably made of a firm, non-elastic fabric such as cotton canvas. In an embodiment, it can comprise one or more holes to improve ventilation and breathability. In another embodiment, it can be made of a mesh material. The vulvar support may have a triangular cutout in the front to increase flexibility and comfort.

In an embodiment, the side of the vulvar support that is facing the patient's body is lined with a soft material. In another embodiment, there is a pocket on the side of the vulvar support that is facing the patient's body, and a soft pad can be inserted into the pocket to increase the pressure on the vulva. The soft pad can be disposable or reusable/washable. The soft pad can also be made of a coolable gel to provide cooling comfort to the patient.

### BRIEF DESCRIPTION OF THE DRAWINGS

FIG. 1 shows the preferred embodiment of the invention, laid flat.

FIG. 2 shows the preferred embodiment of the invention being worn by a patient.

FIG. 3 shows the preferred embodiment of a fastener.

FIG. 4 shows an alternate embodiment of the present invention, showing a cutout in the front.

FIG. 5 shows an alternate embodiment of the present invention, showing a strap connecting the two rear straps.

FIG. 6 shows an alternate embodiment of the present invention, showing a pocket on the inner side of the vulvar support.

### DETAILED DESCRIPTION OF THE INVENTION

A preferred embodiment is described below. The disclosure is not meant to limit the invention to the below-described embodiment, but includes all other embodiments that a person of reasonable skill in the art would understand to encompass the invention.

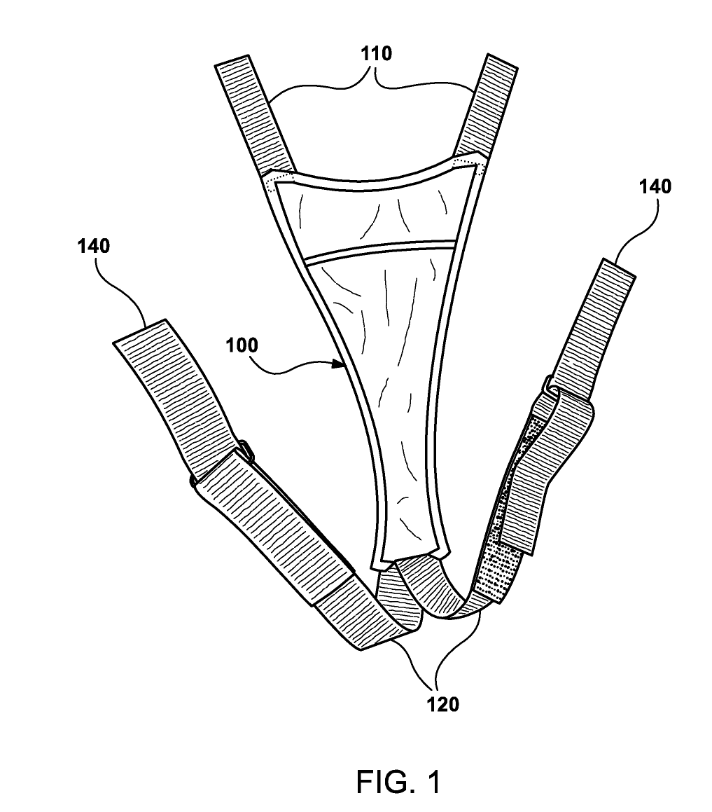

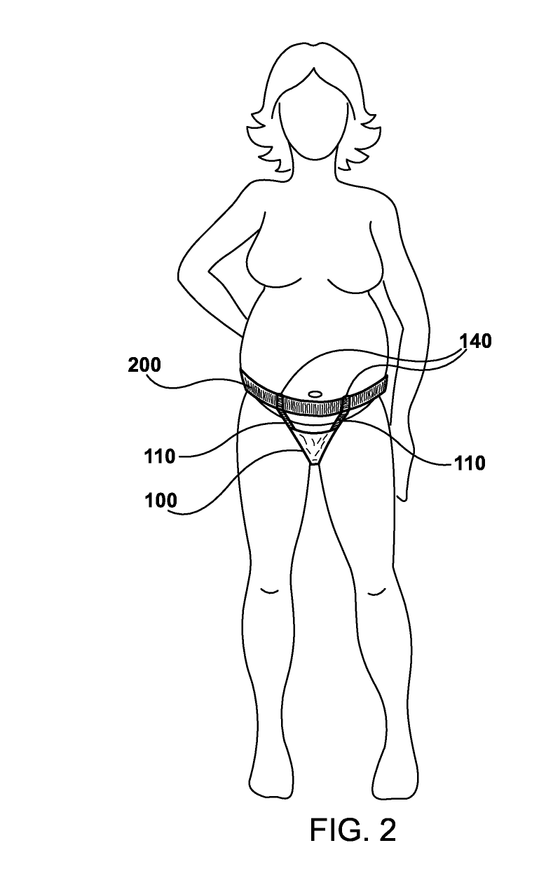

FIG. 1 shows the preferred embodiment of the present invention, laid flat. It comprises a vulvar support 100, front straps 110, and rear straps 120. The front and rear straps 110 and 120 are used to attach the vulvar support 100 to a standard maternity support belt by means of fasteners 140. The vulvar support is preferably made of a strong non-elastic fabric such as cotton canvas, and shaped in such a way as to fit in the patient's crotch and cover the vulvar area. The straps are preferably made of nylon or similar material typically used for making straps. FIG. 2 shows the preferred embodiment of the present invention worn by a patient who is also wearing a maternity support belt 200.

The front and rear straps 110 and 120 preferably comprise Velcro fasteners 140 that attach to corresponding Velcro fasteners on the maternity support belt. Other fasteners may also be used, such as snaps, buttons, hooks, zippers, or any other fastener that can attach a strap to a belt in such a way that the strap can sustain a significant amount of force.

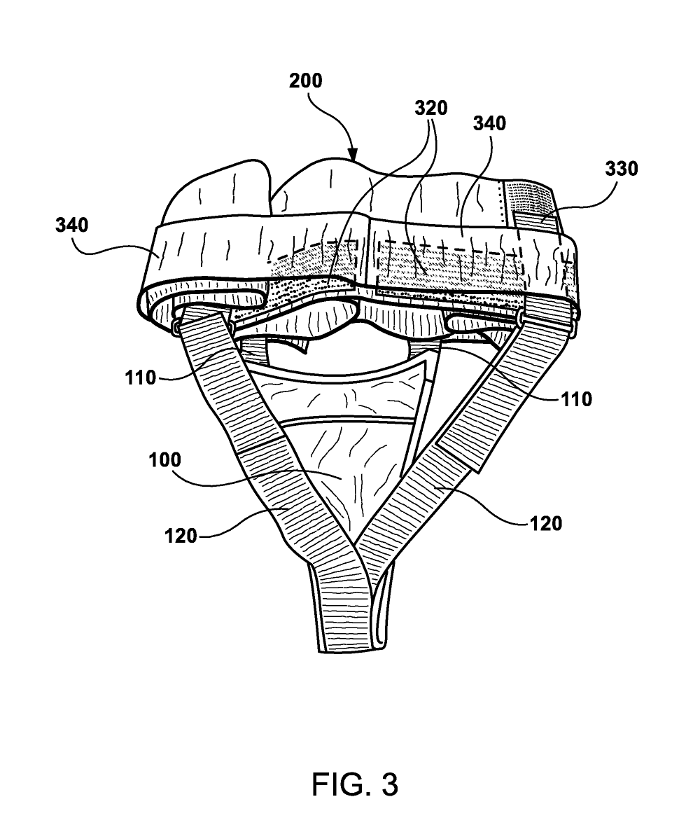

FIG. 3 shows the preferred embodiment of a Velcro fastener 140, shown in detail. The end of the strap comprises an inner Velcro fastener (not shown), which is attached to a corresponding inner Velcro strip on the maternity support belt 320, and an outer Velcro fastener 330 on the other side of the end of the strap. After the inner Velcro fastener is attached to the inner Velcro strip, an outer Velcro strip 340 is attached to the outer Velcro fastener 330 and to the inner Velcro strip 320 on the maternity support belt. This anchors the strap to the maternity support belt in a secure way and allows it to provide more upward force on the vulvar support.

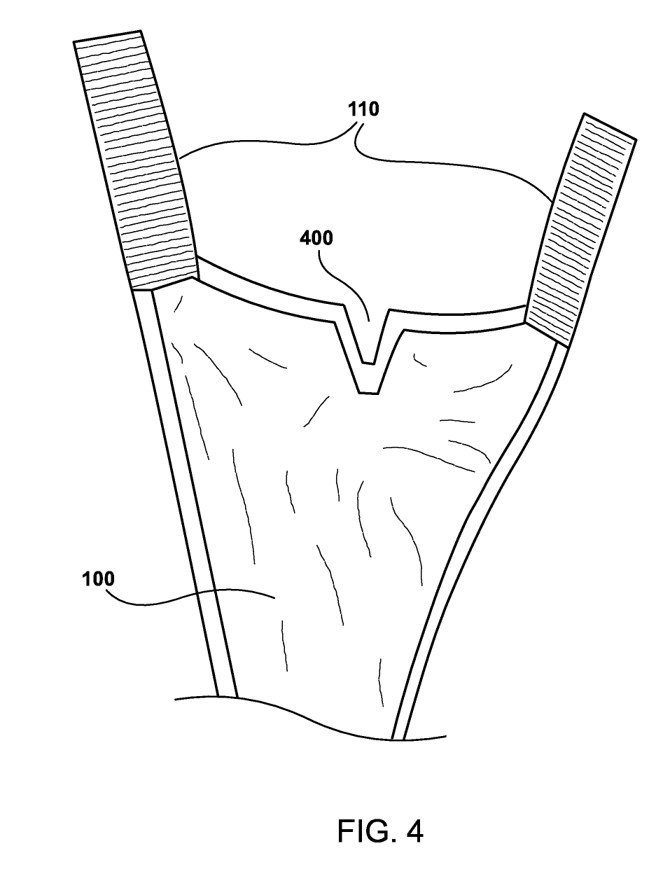

In an embodiment, the vulvar support 100 has a cutout 400 in front, as shown in FIG. 4. This allows for more flexibility in the lateral direction and less chafing and irritation around the edges of the vulvar support. The cutout 400 can be any shape, size, and any angle as long as the vulvar support can still provide the needed pressure to the vulva. In the preferred embodiment, the cutout 400 is triangular, as shown in the Figure.

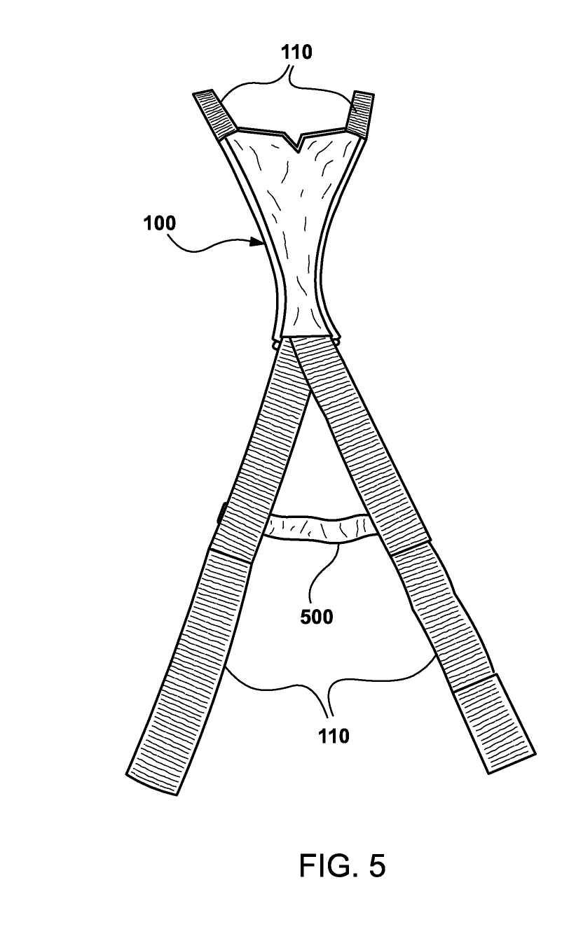

In an embodiment, the two rear straps 120 have a connecting strap 500 between them, as shown in FIG. 5. This prevents them from moving too far apart when the patient moves around or bends or sits down, which may be uncomfortable for the patient. More than one connecting strap may also be used depending on patient needs. The connecting strap or straps 500 preferably attach to the two rear straps 120 by Velcro, but it also may be sewn on permanently, or attached by buttons, snaps, zippers, or any other fastener means known in the art. The connecting strap or straps 500 may be attached at any point along the length of the rear straps, as needed to prevent unnecessary movement.

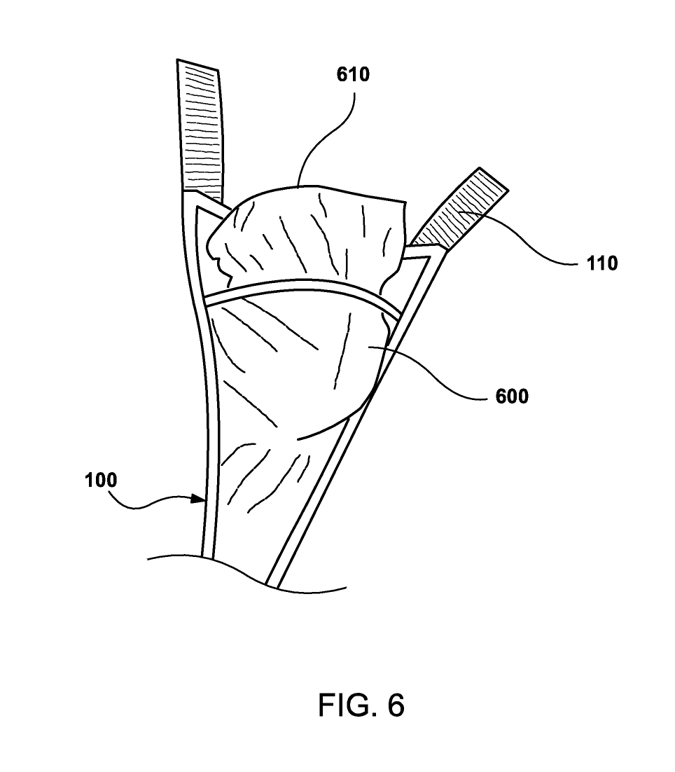

In an embodiment, the vulvar support 100 has a pocket 600 on the side facing the vulva, as shown in FIG. 6. A soft pad 610 may be inserted into the pocket to increase the pressure on the vulva (shown partially inserted in the Figure). Preferably, the soft pad 610 is made of cotton or similar material. The soft pad 610 may be disposable or reusable. The softness or firmness of the soft pad 610, as well as its shape, may be variable depending on patient needs, and a patient may be able to select one of several different pads. The pocket 600 is preferably made of a soft and stretchy material that is comfortable for the patient.

In an embodiment, the soft pad may be made of a gel material that can be cooled to improve patient comfort.

In an alternate embodiment, the soft pad 610 may be attached to the vulvar support 100 by other means, such as Velcro, snaps, hooks, or buttons, rather than inserted into a pocket.

In an embodiment, the vulvar support 100 has a soft lining to improve patient comfort. The soft lining may be flannel, cotton, fleece, or any other material that is soft and comfortable for the patient.

In an embodiment, the vulvar support 100 may have a hole or holes in order to improve ventilation for the vulva. It may also be made of a mesh material to improve breathability.

The present invention is preferably designed to attach to a maternity support belt, since pregnant women are likely to require both a maternity support belt and a vulvar varicosity supporter. However, the present invention may also attach to a standard back brace or any other brace or supporter that goes around the user's waist and is sturdy enough to support the forces put on it by the vulvar varicosity supporter.

1. A supporter for vulvar varicosities, comprising:
a vulvar support, said support shaped in such a way as to comfortably fit in a patient's crotch and vulvar area, said support made of a strong non-elastic fabric, said support comprising a front end and a back end;
a plurality of straps, with at least one front strap attached to the vulvar support at the front end and at least one rear strap attached to the vulvar support at the back end;
fasteners attached to each one of the plurality of straps in such a way as to enable the straps to be attached to a maternity support belt.

2. The supporter for vulvar varicosities of claim 1, wherein the fasteners are one or more of the following group: Velcro fasteners, snaps, buttons, hooks, zippers.

3. The supporter for vulvar varicosities of claim 1, wherein the straps are adjustable in length.

4. The supporter for vulvar varicosities of claim 1, wherein the vulvar support has a triangular cutout at the front end.

5. The supporter for vulvar varicosities of claim 1, comprising two rear straps attached to the vulvar support at the back end, and further comprising a connecting strap attached between the two rear straps.

6. The supporter for vulvar varicosities of claim 1, wherein a soft pad is attached to the vulvar support on a side of the vulvar support facing a patient's vulva when in use.

7. The supporter for vulvar varicosities of claim 6, wherein the vulvar support comprises a pocket on a side of the vulvar support facing a patient's vulva when in use, wherein the soft pad is attached to the vulvar support by being inserted into the pocket.

8. The supporter for vulvar varicosities of claim 6, wherein the soft pad is made of cotton.

9. The supporter for vulvar varicosities of claim 6, wherein the soft pad is disposable.

10. The supporter for vulvar varicosities of claim 6, wherein the soft pad is reusable.

11. The supporter for vulvar varicosities of claim 6, wherein the soft pad is made of a gel material that can be cooled prior to use.

12. The supporter for vulvar varicosities of claim 1, wherein the vulvar support has a soft lining.

13. The supporter for vulvar varicosities of claim 1, wherein the vulvar support comprises at least one hole.

14. The supporter for vulvar varicosities of claim 1, wherein the vulvar support is made of a mesh material.

15. The supporter for vulvar varicosities of claim 1, wherein the vulvar support is made of cotton canvas.
